(19) United States
(12) Patent Application Publication
(10) Pub. No.: US 2022/0095967 A1
(43) Pub. Date: Mar. 31, 2022

(54) SENSOR-COUPLED MICRONEEDLE ARRAY FOR INTERSTITIAL FLUID ANALYSIS

(71) Applicant: X Development LLC, Mountain View, CA (US)

(72) Inventors: Anupama Thubagere Jagadeesh, Palo Alto, CA (US); Benoit Schillings, Los Altos Hills, CA (US); Stefan Bogdanovic, Mountain View, CA (US)

(21) Appl. No.: 17/397,985

(22) Filed: Aug. 9, 2021

Related U.S. Application Data
(60) Provisional application No. 63/085,758, filed on Sep. 30, 2020.

(51) Int. Cl. A61B 5/145; A61B 5/1459

(52) U.S. Cl. CPC A61B 5/14514; A61B 5/14532; A61B 5/14546; A61B 5/1459

(57) ABSTRACT

An apparatus for analysis of interstitial fluid, the apparatus including an array of needles spaced apart from each other; an array of chambers separated from each other and each in fluid communication with a lumen of a corresponding one of the needles, the array of chambers comprising one or more first chambers and one or more second chambers, the one or more first chambers each containing nanoparticles functionalized to bind with a first biomarker present in the interstitial fluid, the one or more second chambers each containing nanoparticles functionalized to bind with a second biomarker present in the interstitial fluid, the first and second biomarkers being different; and an optical sensor array positioned to simultaneously detect light emitted from each of the one or more first and second chambers in response to the first or second biomarkers binding with the nanoparticles.

### CLAIM OF PRIORITY

This application claims priority under 35 USC § 119(e) to U.S. Patent Application Ser. No. 63/085,758, filed on Sep. 30, 2020, the entire contents of which are hereby incorporated by reference.

### FIELD OF THE DISCLOSURE

The disclosure relates to a device for the analysis of target analytes present in the interstitial fluid of a subject.

### BACKGROUND

Quantification of biomarkers found in blood, urine, saliva, and other bodily fluids allows a clinician to diagnose and monitor diseases and biological processes occurring in a patient. A rich source of biomarkers in the body is the interstitial fluid that surrounds cells and tissues in the body. Dermal interstitial fluid is a thin layer of fluid surrounding the dermal cells of a patient, consisting of a water solvent containing sugars, salts, fatty and amino acids, coenzymes, hormones, neurotransmitters, white blood cells and cell waste-products. This fluid and constituent products leak out of the cells and blood capillaries near the surface of the dermis, beneath the epidermis. Difficulty in accessing this fluid has limited its use in research and medicine.

Microneedles are microscopic applicators for delivering vaccines or other drugs across various barriers, including transdermal application. Microneedles range in size, shape, and function and function as an alternative to other delivery methods such as conventional hypodermic needles. The arrays are a collection of microneedles, ranging from only a few microneedles to several hundred. The manufacturer then bonds the microneedle array to an applicator, or other solid stamping device, which allows a clinician to apply the microneedle array to an area of a patient, or a patient to self-administer. The shallow penetration depth of microneedles allow access to the area in which interstitial fluid is found in the skin, between the epidermis and dermis.

### SUMMARY

In general, the disclosure relates to a device for the analysis of biomarkers present in the interstitial fluid of a subject (e.g., a human user). In some examples, the device includes an array of microneedles that, when applied to the dermis of a user, connects an array of chambers to the interstitial fluid of a user via a central micro-capillary channel in each microneedle. Each chamber of the array includes nanoparticles labelled to interact specifically or non-specifically with a particular biomarker.

A user or clinician administers the microarray by placing the device on the epidermis, thereby penetrating the epidermal layer with the microneedles. Interstitial fluid is drawn into a channel within each microneedle via micro-capillary wicking action. In some implementations, the interstitial fluid can then be further drawn into the connected chamber via vacuum. The labelled nanoparticles present in the chamber bind to the biomarker of interest and the binding induces a luminescent reaction, thereby presenting an optical signal.

Alternatively, the nanoparticles can be suspended in a fluid contained within the chamber and the nanoparticles allowed to react with the biomarkers within the chamber or within the micro-capillary channel.

The array includes an optical sensor (e.g., a CMOS sensor) and associated readout device for detection of the optical signals presented by the interactions in each chamber of the array. The sensor monitors each chamber for a signal, and outputs changes in luminescence to a readout device. The user or clinician interacts with the readout device to determine concentration, quantity, or presence of individual or collected biomarkers in the interstitial fluid of the user.

In some implementations, the biomarkers of interest can be biomarkers the body produces naturally, or produced in response to a stimulus, e.g., an immune response, or a drug response. The results can be recorded and analyzed to determine the particular biomarker environment of a user.

In general, in a first aspect, the invention features an apparatus for analysis of interstitial fluid, the apparatus including an array of needles spaced apart from each other; an array of chambers separated from each other and each in fluid communication with a lumen of a corresponding one of the needles, the array of chambers including one or more first chambers and one or more second chambers, the one or more first chambers each containing nanoparticles functionalized to bind with a first biomarker present in the interstitial fluid, the one or more second chambers each containing nanoparticles functionalized to bind with a second biomarker present in the interstitial fluid, the first and second biomarkers being different; an optical sensor array positioned to simultaneously detect light emitted from each of the one or more first and second chambers in response to the first or second biomarkers binding with the nanoparticles.

Embodiments may include one or more of the following features. The array of chambers can include at least one or more additional chambers containing nanoparticles functionalized to bind with one or more respective biomarkers different from the first and second biomarkers. Each needle can have a bore with a diameter sufficiently narrow to draw interstitial fluid from the lumen to the corresponding chamber by capillary action at STP. The first and second biomarkers can be selected from the group including a red blood cell biomarker, a white blood cell biomarker, a sodium biomarker, a potassium biomarker, a magnesium biomarker, a glucose biomarker, a vitamin a biomarker, a vitamin d biomarker, an iron biomarker, a hemoglobin biomarker, a melanin biomarker, a carotenoid biomarker, a cytokine biomarker, and an interleukin biomarker. The apparatus can include an electronic processing apparatus in communication with the optical sensor array, the electronic processing apparatus being programmed to analyze signals from the optical sensor array to determine information about the interstitial fluid.

Among other advantages, cost-effective methods of manufacturing the microneedle arrays for the interstitial fluid analysis device leads to lower cost per application. Further, the microneedle arrays include tens to hundreds of individual needles, each connected to a discrete chamber. Each chamber includes a unique nanoparticle for binding a specific biomarker, therefore the array detects the same number of biomarkers as chambers per application. Including each unique nanoparticle in a distinct chamber spatially isolates each binding reaction, reducing interactions between nanoparticles. Further, each chamber being spatially separate creates uniquely addressable locations to gather spatial information on the interstitial fluid and allowing multiple reactions to be run in parallel, greatly increasing testing throughput.

Conventional drug delivery or sample collection devices (e.g., hypodermic needles) can induce significant stress reactions in a patient, thereby altering the overall composition of the biomarkers within the interstitial fluid. Performing the sample collection and testing with microneedle arrays that penetrate the epidermis without disrupting nerve endings in the dermis reduces stress reactions in the patient and allows for collection of interstitial fluid representative of the basal state of a patient.

Other advantages will be apparent from the description, the drawings, and the claims.

### BRIEF DESCRIPTION OF THE DRAWINGS

FIG. 1 is a cross-sectional schematic view detailing the primary components of an exemplary interstitial fluid analysis device.

FIG. 2 A is a cross-sectional schematic view of the exemplary microneedle array of FIG. 1.

FIGS. 2B and 2C are schematic diagrams of further exemplary embodiments of microneedle arrays.

FIG. 3 is a cross-sectional schematic view of the exemplary interstitial fluid analysis device applied to the skin of a user.

FIG. 4 is a schematic diagram of an exemplary readout device.

In the figures, like symbols indicate like elements.

### DETAILED DESCRIPTION

This disclosure describes an interstitial fluid (IF) analysis device including a microneedle array, an array of connected chambers, and an optical sensor array for the detection of optical signals indicating the presence of one or more biomarkers present in the IF of a user. Hypodermic needles used by clinicians generally penetrate deep into subdermal layers to access veins or musculature before taking samples or depositing solutions. The hypodermic needles are also generally wide (<450 μm) and long (<1 cm) compared to dermal cellular structures (˜1-100 μm), reaching and disrupting nerve endings in the dermal layers before reaching their destination, triggering a pain response in a patient.

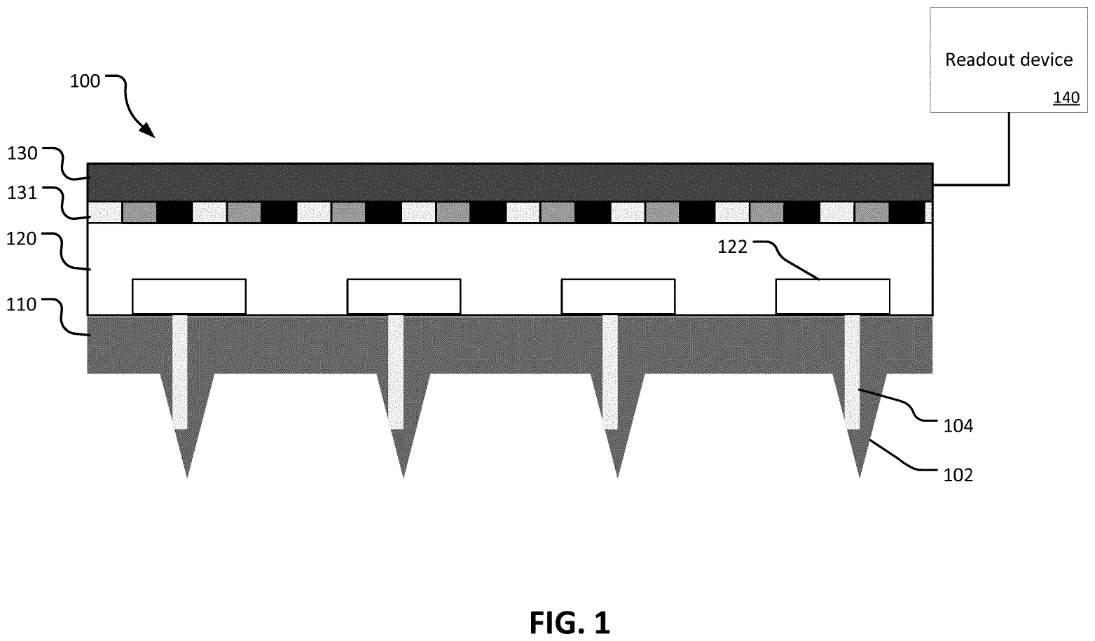

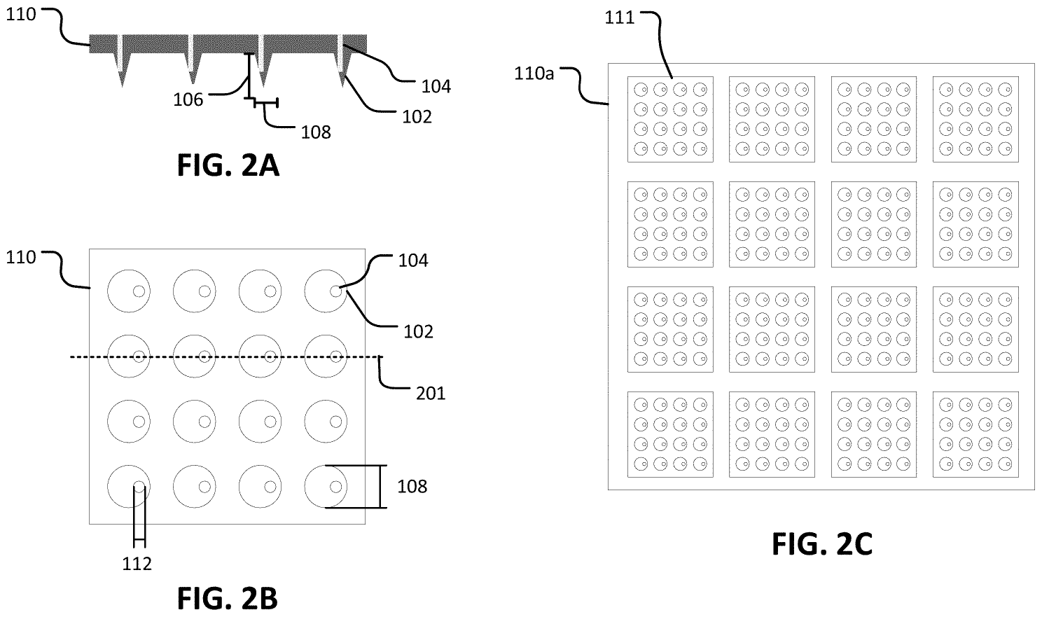

The microneedle array of an IF analysis device is composed of a number of static microneedles of equal length that penetrate to a shallow depth (<750 μm) when compared to the thickness of the dermis (˜2 mm) thereby avoiding significant damage to nerve endings and structural cells. FIG. 1 depicts the components of the IF analysis device 100 and their respective arrangement. Beginning at the bottom of FIG. 1, the microneedle array 110 is a single body into which individual microneedles 102 are constructed. The array 110 is composed of a material of sufficient rigidity to prevent collapse of the microneedle 102 structure upon application of the IF analysis device 100 and puncturing the skin of a user. For example, the microneedle array 110 can be composed of quartz, silicon, titanium, stainless steel, or polycarbonate. The structure and arrangement of the individual microneedles 102 of an array 110 is shown in FIGS. 2A through 2C.

FIG. 2A is a cross-sectional side view of the microneedle array 110 shown in FIG. 1, including four exemplary microneedles 102 and respective micro-capillary channels 104. The microneedles 102 of an array 110 penetrates the skin of a user and the micro-capillary channel transports (e.g., draws) IF fluid via micro-capillary action from the entrance port on the side surface of a microneedle 102, through the lumen (e.g., bore) of the channel 104, and through the body of the microneedle array 110. The surface opposite the microneedles 102 is planar to form a positive seal with additional components of the IF analysis device 100.

The microneedles 102 and channels 104 of the microneedle array 110 can be constructed via subtractive manufacturing methods. In some implementations, this can include lithographic processes wherein the body is exposed to high intensity light (e.g., photolithography) or an electron beam (e.g., beam lithography) through a mask which directs the removal of unwanted material thereby forming the microneedles 102. A micro-capillary channel 104 is also etched into a surface of each microneedle 102, extending to and oriented orthogonally from the planar surface of the array 110, thereby connecting an exterior surface of microneedle 102 to the opposite surface of the array 110.

Alternatively, the microneedle array 110 can be cast as a single unit, including all microneedles 102, using micromolding methods. For example, an exemplary master copy of the micro microneedle array 110 can be constructed via lithographic etching. The master is then used to cast a silicone blank, having the inverted structure of the master. Liquid polymer solutions are flown into the mold, filling the voids of the blank and replicating the structure of the master copy of the microneedle array 110. The mold is then removed and the microneedle array 110 hardened via photocrosslinking.

The microneedles 102 are constructed to a uniform length 106 extending from the array 110 at equal spacings. The microneedles 102 can be 5 mm or less in length 106 (e.g., 3 mm or less, 2 mm or less, 1 mm or less, 0.5 mm or less). For example, microneedles 102 less than 500 μm in length 106 can avoid disrupting nerve endings in the dermis, thereby circumventing a pain response.

To produce a tapered, needle-like profile, the width 108 of the microneedles 102 is less than the length 106 and can be 2 mm or less (e.g., 1.5 mm or less, 1 mm or less, 0.5 mm or less). The microneedles 102 of FIG. 2A have a triangular cross-sectional profile and can be conical (e.g., radially symmetric) or pyramidal (e.g., planar sides with a geometric base) in three-dimensional shape. FIG. 2B shows a top-down schematic view of the surface of an exemplary microneedle array 110 including sixteen conical (e.g., radially symmetric) microneedles 102. The dotted line 201 bisecting four microneedles 102 and their respective micro-capillary channels 104 is the cross sectional view depicted in FIG. 2A.

The microneedle array 110 of FIG. 2B depicts a regular array (e.g., equal spacing) of sixteen conical microneedles 102 arranged in an equally-spaced square of four by four microneedles 102. Each microneedle 102 of the array 110 has an equal width 108 and each micro-capillary channel 104 within each microneedle 102 shares an equal diameter 112. The channel 104 of each microneedle 102 is offset from the symmetric center of the microneedle 102 by a distance. The outer dimensions of the array 110 in FIG. 2B are equal (e.g., square), though this is not necessary. For example, a regular array of five by three microneedles can have non-equal outer dimensions (e.g., rectangular).

In general, the array 110 can be a regular array of “m” by “n” needles where “m” and “n” are integer values, for example, the four by four array 110 shown in FIG. 2B. In some implementations, the microneedle array 110 can be an irregular array (e.g., non-equal spacing) of microneedles 102. In some implementations, the microneedles 102 of an array 110 can be manufactured in a number of smaller microneedle sub-arrays, each smaller sub-arrays including a subset of the total number of microneedles 102 in the array 110. The microneedles 102 of the sub-arrays can be regularly or irregularly spaced in their respective groups.

Depicted in FIG. 2C is a second exemplary microneedle array 110a including sixteen subarrays 111, each subarray 111 including sixteen microneedles 102 arranged in a regular array as shown in FIG. 2B.

Referring again to FIG. 1, a chamber housing 120 is disposed above and affixed to the upper planar surface of the microneedle array 110. The chamber housing 120 is a transparent body into which an array of chambers 122 is constructed. The chamber housing 120 is constructed of a material that is substantially transparent at visible, ultraviolet, or infrared wavelengths such as glass, quartz, acrylic, or other rigid plastics. Subtractive manufacturing methods, such as lithographic processes, are used to construct the chambers 122 into the housing 120. Alternatively, the chamber housing 120 can be cast using micromolding methods.

The length and width of the chamber housing 120 is constructed to match the length and width of the microneedle array 110. The chambers 122 are geometric voids in one surface of the chamber housing 120 into which IF fluid will be drawn when the IF analysis device is applied to the skin of a user. FIG. 1 depicts the cross-sectional profile of the chambers 122 as rectangular, but this is not necessary. The shape, height, and width of the chambers 122 are sufficient to create an interior volume of about 500 μl or less (e.g., 400 μl or less, 200 μl or less, 100 μl or less, 50 μl or less). The maximum lateral dimension of a chamber 122 is less than the center-to-center spacing of the microneedles 102 of the microneedle array 110 such that no two chambers are in fluid connection.

Each chamber 122 of the housing 120 is constructed in the same array pattern as the microneedles 102 of the microneedle array 110. In this manner, when the housing 120 is affixed in register with the microneedle array 110, a chamber 122 of the chamber housing 120 aligns with each microneedle 102 of the microneedle array 110. In this configuration, any sample drawn through the lumen of a micro-capillary channel 104 of a microneedle 102 accesses only a single corresponding chamber 122.

In some implementations, the chambers housing 120 affixes to the microneedle array 110 in such a manner as to create and maintain a reduced pressure (e.g., partial vacuum, or less than 1 atm) within the chambers 122, aiding the micro-capillary drawing action of the channel 104. For example, the chamber housing 120 can be affixed with an inorganic glue to the microneedle array 110 in a vacuum chamber.

Affixed to the surface of the chamber housing 120 opposing the microneedle array 110 is an optical sensor array 130. The optical sensor array 130 is a digital image sensor for detecting light emissions from the chemical reactions taking place in the chambers 122. The optical sensor array 130 shares the same length and width as the chamber housing 120 and the microneedle array 110 to detect emissions from every chamber 122 of the array.

In some implementations, the optical sensor array 130 is a complementary metal oxide semiconductor (CMOS) sensor with an affixed color filter array 131. A CMOS sensor can provide high noise immunity, high readout rate, increased dynamic range, and/or low power consumption among various advantages.

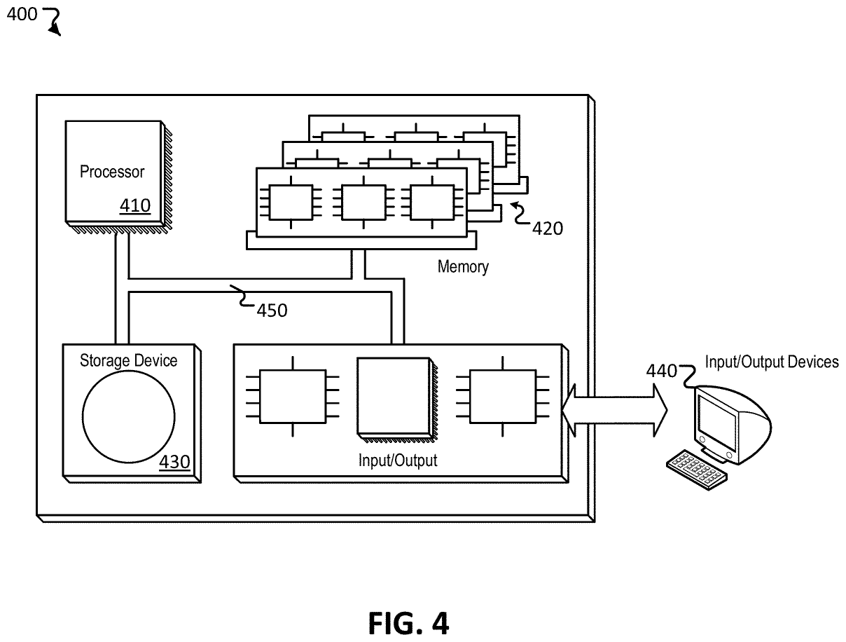

The optical sensor array 130 is in electronic communication with a connected electronic processing apparatus, e.g., readout device 140, for displaying collected image information. An exemplary readout device is shown in FIG. 4.

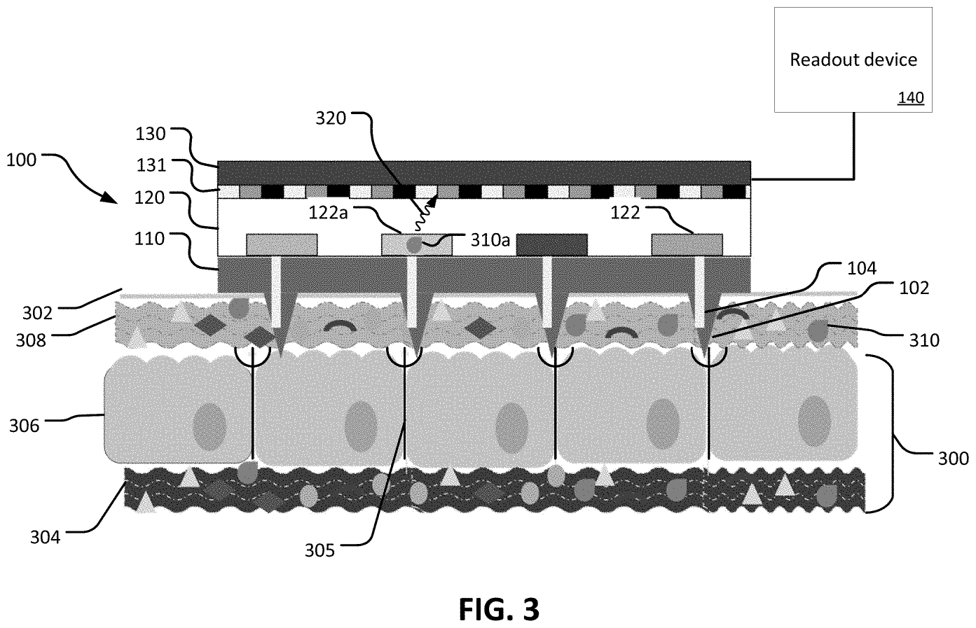

The IF analysis device 100 is used for the determination of the presence of biomarkers in the IF fluid occupying the space between the outermost two layers of skin, the dermis and epidermis of a user. FIG. 3 depicts a cross-sectional view of the IF analysis device 100 applied to the skin of a user. The epidermis 302 is the outermost layer of cells layered above the dermis 300 of a user, the dermis 300 being a layer of tissue, containing blood capillaries 304, nerve endings 305, sweat glands, a base layer 306 of columnar cells (e.g., the stratum basale), and other structures.

Capillaries 304 are semi-permeable barriers carrying and exchanging a number of biological compounds around the body, including to and from the dermis 300. These compounds commonly include oxygen, nutrients, metabolic waste, and biomarkers 310 from other areas of the body. Biomarkers 310 are chemical indicators of a biological process occurring in a body, and can include indicators for a particular disease or physiological state. During the transport and exchange of these compounds, biomarkers 310 pass from the capillaries 304, through the base layer 306, and into the interstitial (IF) fluid 308 that pervades the dermis 300.

Examples of biomarkers found in IF fluid 308 can include red blood cells, white blood cells, platelets, sodium, potassium, magnesium, nitrogen, carbon dioxide, oxygen, glucose, Vitamin A, Vitamin D, Vitamin B1 (thiamine), Vitamin B12, folate, calcium, Vitamin E, Vitamin K, zinc, copper, Vitamin B6, Vitamin C, homocysteine, iron, hemoglobin, hematocrit, insulin, melanin, hormones, testosterone, estrogen, cortisol, thyroxine, triiodothyronine, human growth hormone, insulin-like growth factors, thyroid stimulating hormone (TSH), carotenoids, cytokines, interleukins, chlorides, cholesterols, lipoproteins, triglycerides, c-peptide, creatinine, creatine, creatine kinase, urea, ketones, peptides, proteins, albumin, bilirubin, myoglobin, ESR, CRP, IL6, immunoglobins, resistin, ferritin, transferrin, antigens, troponins, gamma-glutamyltransferase (GGT), lactate dehydrogenase (LD), alanine aminotransferase, alkaline phosphatase, and aspartate aminotransferase.

The extracellular IF fluid 308 found outside of the capillaries 304 perfuses the region between the layers of the epidermis 302 and the underlying cells of the base layer 306. FIG. 3 depicts a number of compounds within the IF fluid 308 including a number of biomarkers 310. A microneedle array 110 applied to the skin of a user penetrates the epidermis 302 and provides a means to access the IF fluid 308 without disrupting nerve endings 305 within the base layer 306 of the dermis 300, thereby avoiding an induced pain response for the user.

The microneedle array 110 of an IF analysis device 100 applied to the skin punctures the epidermis 302 and enters the area above the base layer 306 of the dermis 300 containing the IF fluid 308. The micro-capillary channel 104 of each microneedle 102 draws a sample of the IF fluid 308 into the chamber via capillary action. In some implementations, reduced pressure in the corresponding chamber 122 further draws the sample of the IF fluid 308 into the chamber 122.

Each chamber 122 in fluid communication with a corresponding microneedle 102 includes a labelled nanoparticle for specifically binding and reporting the presence of a biomarker 310 of interest. Examples of nanoparticles can include organic (e.g., DNA origami) or inorganic (e.g., gold) nanoparticles. For example, inorganic nanoparticles can be prepared from metals, (e.g., iron, gold, and silver), inorganic salts, and ceramics (e.g., phosphate or carbonate salts of calcium, magnesium, or silicon), magnetic nanoparticles, fullerenes (e.g., soluble carbon molecules), three-dimensional carbon structure (e.g., nanotubes). Examples of organic nanoparticles can include liposomes, micelles, protein/peptide structures, and branched polymers.

The nanoparticles can be labelled through covalent or non-covalent attachment of a labelling molecule. Labelling molecules can include antibodies, antigens, peptides, DNA, RNA, proteins, recombinant proteins, fluorescent molecules, or chemiluminescent molecules. The surface of a nanoparticle can be coated to facilitate binding of the biomarker 310 (e.g., antibodies), or the surface can be chemically modified to facilitate attachment of the biomarker 310 (e.g., biotinylated).

Once a chamber 122 is exposed to a sample of IF fluid 308 containing the biomarker 310 the nanoparticle within the chamber 122 binds to the biomarker 310 and undergoes a chemical reaction that results in the emission of an optical signal 320 (e.g., luminescence), thereby signaling the presence of the biomarker 310 in the chamber 122. FIG. 3 depicts chamber 122a containing labelled nanoparticles bound to biomarker 310a and emitting an optical signal 320. In some implementations, an optical signal 320 can constitute the cessation of emission (e.g., quenching) when the nanoparticle binds to a biomarker 310.

IF fluid 308 found within the dermis 300 of a user contains a large collection of biomarkers 310 in solution. In general, each chamber 122 of the IF analysis device 100 contains a unique nanoparticle for the detection of a discrete biomarker 310. In some implementations, a chamber 122 can have more than one unique labelled nanoparticle for the detection of a corresponding number of biomarkers 310 within a chamber 122.

In some implementations, the labelled nanoparticle is affixed to one or more inner surfaces of chamber 122. In some implementations, the labelled nanoparticle is affixed to the interior surface of the micro-capillary channel 104. For example, labelled nanoparticles can be affixed through a biotin-streptavidin chemical linkage. In some implementations, the labelled nanoparticle is in a solution contained within the chamber 122.

The labelled nanoparticles bound to biomarkers 310 in a chamber 122 emit an optical signal 320 (e.g., at least one photon). The optical signal 320 travels through the transparent chamber housing 120 and strikes the optical sensor 130 on at least one pixel. The optical sensor 130 detects the presence, intensity, and pixel location(s) of the optical signal 320 and converts the optical signal 320 to an electronic signal, e.g., an image. The image is read out from the optical sensor 130 and transmitted to the readout device for display to and interpretation by the user or clinician. In some implementations, the readout device can use a wired or wireless connection protocol to communicate with the optical sensor 130, e.g., Wi-Fi, near field communication (NFC), or Bluetooth®.

The readout device 140 can collect and interpret the transmitted image information according to one or more programs contained on the readout device 140. The readout device 140 can display the information to the user through one or more charts detailing the presence of, type, quantity, concentration, isoform, or spatial location within the array of at least one biomarker 310 present in a chamber 122 of the IF analysis device 100. In this manner, the profile of a number of biomarkers 310 at least equal to the number of microneedles 102 and corresponding chambers 122 in the IF fluid 308 of a user can be analyzed. Further implementations of the readout device 140 are described in FIG. 4.

In some implementations, the microneedle array 110 is constructed using methods described above from optically transparent materials such as acrylic, polycarbonate, silicon dioxide or quartz. In such examples, nanoparticles are affixed to the exterior surface of the microneedle 102 and each microneedle 102 of the array 110 includes a unique nanoparticle. When a user applies the optically transparent microneedle array 110 and punctures the epidermis 302, interstitial fluid 308 surrounds the microneedles 102 and the labelled nanoparticles affixed to the surface of the microneedles 102 bind to respective biomarkers 310. In this implementation, IF analysis device 100 does not include chamber housing 120 as reactions occur on the external surface of the microneedle array 110.

Bound labelled nanoparticles emit an optical signal 320 which enters the transparent microneedle array 110. The shape of the microneedles 102 collimates the optical signal 320 and directs the signal toward the optical sensor 130, performing as an optical waveguide. The optical signal 320 travels through the transparent chamber housing 120 and strikes the optical sensor 130 on at least one pixel. The optical sensor 130 detects the presence, intensity, and pixel location(s) of the optical signal 320 and converts the optical signal 320 to an electronic signal which is readout from the optical sensor 130 and transmitted to the readout device.

FIG. 4 is a schematic diagram of an exemplary readout device 400. The device 400 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., device 400) and their structural equivalents, or in combinations of one or more of them. The device 400 is intended to include various forms of digital computers, such as laptops, desktops, workstations, personal digital assistants, servers, blade servers, mainframes, and other appropriate computers, including vehicles installed on base units or pod units of modular vehicles. The device 400 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The device 400 includes a processor 410, a memory 420, a storage device 430, and an input/output device 440. Each of the components 410, 420, 430, and 440 are interconnected using a system bus 450. The processor 410 is capable of processing instructions for execution within the device 400. The processor may be designed using any of a number of architectures. For example, the processor 410 may be a CISC (Complex Instruction Set Computers) processor, a RISC (Reduced Instruction Set Computer) processor, or a MISC (Minimal Instruction Set Computer) processor.

In one implementation, the processor 410 is a single-threaded processor. In another implementation, the processor 410 is a multi-threaded processor. The processor 410 is capable of processing instructions stored in the memory 420 or on the storage device 430 to display graphical information for a user interface on the input/output device 440.

The memory 420 stores information within the device 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the device 400. In one implementation, the storage device 430 is a computer-readable medium. In various different implementations, the storage device 430 may be a floppy disk device, a hard disk device, an optical disk device, or a tape device.

The input/output device 440 provides input/output operations for the device 400. In one implementation, the input/output device 440 includes a keyboard and/or pointing device. In another implementation, the input/output device 440 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

A number of embodiments have been described. Other embodiments are in the following claims.

What is claimed is:

1. An apparatus for analysis of interstitial fluid, the apparatus comprising:
an array of needles spaced apart from each other;
an array of chambers separated from each other and each in fluid communication with a lumen of a corresponding one of the needles, the array of chambers comprising one or more first chambers and one or more second chambers, the one or more first chambers each containing nanoparticles functionalized to bind with a first biomarker present in the interstitial fluid, the one or more second chambers each containing nanoparticles functionalized to bind with a second biomarker present in the interstitial fluid, the first and second biomarkers being different; and
an optical sensor array positioned to simultaneously detect light emitted from each of the one or more first and second chambers in response to the first or second biomarkers binding with the nanoparticles.

2. The apparatus of claim 1, wherein the array of chambers comprises at least one or more additional chambers containing nanoparticles functionalized to bind with one or more respective biomarkers different from the first and second biomarkers.

3. The apparatus of claim 1, wherein each needle has a bore with a diameter sufficiently narrow to draw interstitial fluid from the lumen to the corresponding chamber by capillary action at standard temperature and pressure (STP).

4. The apparatus of claim 1, wherein the first and second biomarkers are selected from the group consisting of a red blood cell biomarker, a white blood cell biomarker, a platelet biomarker, a sodium biomarker, a potassium biomarker, a magnesium biomarker, a nitrogen biomarker, a carbon dioxide biomarker, an oxygen biomarker, a glucose biomarker, a Vitamin A biomarker, a Vitamin D biomarker, a Vitamin B1 (thiamine) biomarker, a Vitamin B12 biomarker, a folate biomarker, a calcium biomarker, a Vitamin E biomarker, a Vitamin K biomarker, a zinc biomarker, a copper biomarker, a Vitamin B6 biomarker, a Vitamin C biomarker, a homocysteine biomarker, an iron biomarker, a hemoglobin biomarker, a hematocrit biomarker, an insulin biomarker, a melanin biomarker, a hormone biomarker, a testosterone biomarker, an estrogen biomarker, a cortisol biomarker, a thyroxine biomarker, a triiodothyronine biomarker, a human growth hormone biomarker, an insulin-like growth factors biomarker, a thyroid stimulating hormone (TSH) biomarker, a carotenoid biomarker, a cytokine biomarker, a interleukin biomarker, a chloride biomarker, a cholesterol biomarker, a lipoprotein biomarker, a triglyceride biomarker, a c-peptide biomarker, a creatinine biomarker, a creatine biomarker, a creatine kinase biomarker, a urea biomarker, a ketone biomarker, a peptide biomarker, a protein biomarker, a albumin biomarker, a bilirubin biomarker, a myoglobin biomarker, a erythrocyte sedimentation rate (ESR) biomarker, a C-reactive protein (CRP) biomarker, an Interleukin-6 (IL6) biomarker, an immunoglobin biomarker, a resistin biomarker, a ferritin biomarker, a transferrin biomarker, an antigen biomarker, a troponin biomarker, a gamma-glutamyltransferase (GGT) biomarker, a lactate dehydrogenase (LD) biomarker, an alanine aminotransferase (ALT) biomarker, an alkaline phosphatase (ALP) biomarker, and an aspartate aminotransferase (AST) biomarker.

5. The apparatus of claim 1, further comprising an electronic processing apparatus in communication with the optical sensor array, the electronic processing apparatus being programmed to analyze signals from the optical sensor array to determine information about the interstitial fluid.

6. The apparatus of claim 5, wherein the optical sensor array comprises a complementary metal oxide semiconductor (CMOS) sensor.

7. The apparatus of claim 1, wherein the needles are 5 mm or less in length.

8. The apparatus of claim 1, wherein the array of needles comprises 256 needles or less.

9. The apparatus of claim 1, wherein each chamber of the array of chambers comprises a volume of 500 μl or less.

10. The apparatus of claim 1, wherein the nanoparticles comprise inorganic nanoparticles or organic nanoparticles.

11. The apparatus of claim 10, wherein the inorganic nanoparticles are selected from the group consisting of metals, inorganic salts, ceramics, magnetic nanoparticles, fullerenes, or three-dimensional carbon structure.

12. The apparatus of claim 10, wherein the organic nanoparticles are selected from the group consisting of comprising liposomes, micelles, protein/peptide structures, and branched polymers.

13. The apparatus of claim 1, wherein the nanoparticles are labelled with labelling molecules comprising one or more antibodies, antigens, peptides, DNA, RNA, proteins, recombinant proteins, fluorescent molecules, or chemiluminescent molecules.

14. A method for detecting a presence of a biomarker in interstitial fluid of a user, comprising:
inserting an array of needles of into a skin of a user;
drawing a sample of interstitial fluid (IF) through the needles of the array of needles into an array of chambers, the array of chambers comprising a nanoparticle functionalized to bind with a first biomarker present in the interstitial fluid; and
detecting light emitted from each of the chambers of the array of chambers in response to the first biomarker binding with the nanoparticles.

15. The method of claim 14, wherein the array of needles is inserted to a depth of 750 μm or less.

16. The method of claim 14, wherein the drawing is performed by micro-capillary action.

17. The method of claim 14, wherein the array of chambers comprises a reduced pressure aiding the drawing of the sample of IF fluid.

18. The method of claim 14, further comprising converting the detected light to an electronic signal, and transmitting the electronic signal to a readout device for display.

19. The method of claim 18, further comprising displaying information detailing a presence of, type, quantity, concentration, isoform, and/or spatial location within the array of needles of the biomarker present in a chamber.

20. The method of claim 14, wherein the inserting is performed by the user.
